(19) United States
(12) Patent Application Publication
(10) Pub. No.: US 2026/0235619 A1
(43) Pub. Date: Aug. 13, 2026

(54) METHOD FOR HOST CELL PROTEIN DETECTION

(71) Applicant: UCB BIOPHARMA SRL, BRUSSELS (BE)

(72) Inventor: SOMAR KHALIL, BRUSSELS (BE)

(21) Appl. No.: 19/160,570

(22) PCT Filed: Feb. 29, 2024
(86) PCT No.: PCT/EP2024/055233

§ 371 Date: Aug. 28, 2025

(30) Foreign Application Priority Data
Mar. 3, 2023 (EP) 23160057.8

(51) Int. Cl. G01N 33/68; G01N 1/40; G01N 30/72

(52) U.S. Cl. CPC G01N 33/6848; G01N 1/4044; G01N 30/7233

(57) ABSTRACT

The present invention provides method for the measurement of residual host cell proteins (HCPs) in a recombinant protein sample based on the combination of affinity chromatography and a digestion under native conditions, prior to analysis using reversed-phase liquid-chromatography coupled to tandem mass spectrometry (LC/MS-MS), wherein the liquid chromatography is performed in a multistep gradient of charge enhancer concentration.

### FIELD OF THE INVENTION

The current invention pertains to the field of recombinant antibody manufacture, and in particular to the detection of residual host cell protein impurities during purification or in a sample of purified protein.

### BACKGROUND OF THE INVENTION

Biotechnology products, in many cases recombinant proteins although not limited to such, are obtained via complex production systems involving the use of genetically modified host cells (bacteria, yeast or mammalian cells). During the manufacturing of such biotechnology products the host systems such as Chinese hamster ovary (CHO) cells, also express at varying degrees a substantial number of endogenous proteins essential for cellular development and viability, generally referred to as Host Cell Proteins (HCPs) [1]. Whatever the product and production system, residual HCPs have to be tested for on a routine basis, as it is necessary to ensure that such impurities are reduced to an acceptable level when the biotechnological product is destined for medicinal use, and ultimately administration to a patient.

Elimination of HCPs typically makes use of chromatographic columns used during purification of the desired biotechnology product, and is frequently referred to as downstream processing (DSP).

However, it is known for example in the field of recombinant antibody production that a subset of endogenous HCPs has demonstrated a likelihood of co-purification through the different DSP steps and are considered difficult to remove mostly due to their interactions with the antibody or resins, and some of them are generally deemed “high risk” based on their impact [2]. Indeed, such process-related impurities are regarded as critical quality attributes (CQAs) since they may pose a risk to clinical safety, product quality, or efficacy [3], and therefore it is becoming vital to establish analytical procedures capable of identifying and monitoring HCP levels in order to support the risk assessment of HCP impurities across manufacturing batches of therapeutic recombinant antibodies.

Conventionally, the quantification of HCPs has been carried out by applying summative approaches based on enzyme-linked immunosorbent assay (ELISA), utilising antibodies generated against the variety of HCPs expressed in the host cell [4]. However, the ELISA only quantifies the total amount of HCPs and does not indicate the identity or quantity of any specific HCP. Mass spectrometry (MS)-based proteomics applications demonstrate an advantage in quantitatively profiling individual HCPs. However, technical reproducibility, dynamic range, and ensuring an acceptable statistical significance of scoring measurements are critical hurdles that still need to be overcome.

Label-free quantification (LFQ) methodology has grown in popularity since it directly quantifies the signal response of associated peptides to determine the quantity of diverse HCPs in an antibody sample [5]. Label free quantification methods are typically divided into intensity-based measurements and spectral counting measurements. Intensity-based measurements such as Hi3 have grown in popularity and demonstrated better accuracy in comparison to spectral counting when using high-resolution MS.

Another aspect to consider is the type of data acquisition performed on the MS. Data-independent acquisition (DIA) has been considered as an option but has a drawback in the loss of precursor selectivity. Data-dependent acquisition (DDA), is widely used in discovery proteomics and has demonstrated applicability in the field of HCP analysis [6]. However, DDA has dynamic range issues that results in missing values which could impact results' consistency. [7].

Several approaches to tackle the dynamic range issue resulting from the low abundance of HCPs in purified antibodies have been described. Despite their limited throughput, multidimensional LC-MS setups have successfully addressed sample complexity by providing higher sensitivity and lower detection limits. Other methods use affinity chromatography (protein A, G, or L) to capture and deplete the majority of the antibody from the sample. Huang et al [8] described a native digestion approach in which the antibody sample is digested in non-denaturing conditions with a very low enzyme-protein ratio.

High field asymmetric waveform ion mobility spectrometry (FAIMS), affinity depletion, and native digestion were recently combined and found, with a lower limit of detection of 1 ppm, to be more effective than established methods [9].

Therefore there remains a need in the art for methods with sufficient sensitivity to detect HCPs present in samples of recombinant proteins.

In particular, when considering recombinant proteins destined for use as pharmaceuticals, there are specific requirements in terms of purity that must be met in order to release the product on the market. As such, there is a particular need for methods that would be suitable for and/or compliant with quality guidelines such as ICH quality guidelines, in particular ICH Q2 guidelines.

### SUMMARY OF THE INVENTION

Against this background the inventors provide a method for the measurement of residual host cell proteins (HCPs) in a recombinant protein sample based on the combination of affinity chromatography and a digestion under native conditions, prior to analysis using reversed-phase liquid-chromatography coupled to tandem mass spectrometry (LC/MS-MS), wherein the liquid chromatography is performed in a multistep gradient of charge enhancer concentration. Furthermore, the inventors found that utilizing a wash solution during the affinity chromatography step maximizes recovery of difficult to remove HCPs and increases their identification.

### BRIEF DESCRIPTION OF THE FIGURES

FIG. 1. Bar plot of the total HCP protein groups identified by normal digestion, native digestion, Prot A with normal digestion, Prot L with normal digestion, Prot A with native digestion, Prot L with native digestion.

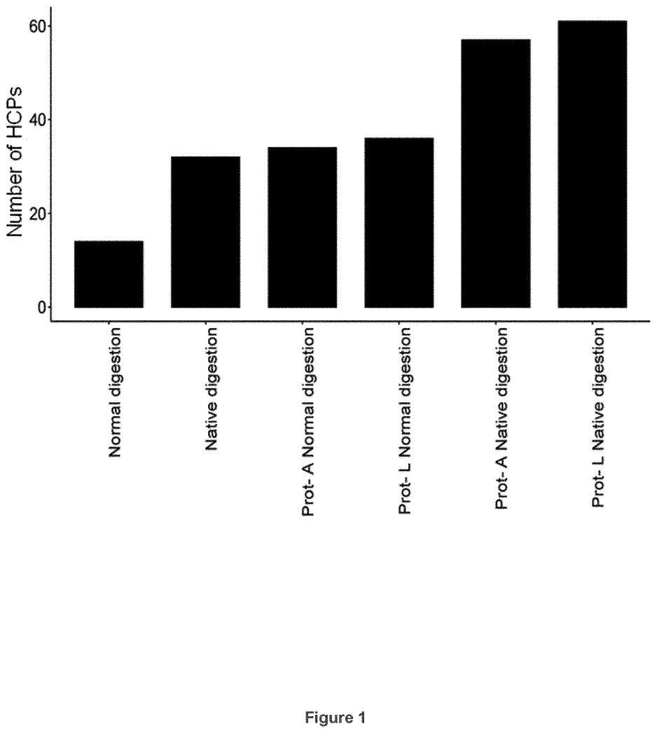

FIG. 2. Bar plot of the total HCP protein groups identified by a combination of Prot A and native digestion. No wash (the resin was only rinsed with the loading buffer), wash with arginine buffer at an alkaline pH, wash with citric acid+sodium citrate buffer at pH 5.5, and wash with arginine+sodium caprylate buffer at alkaline pH.

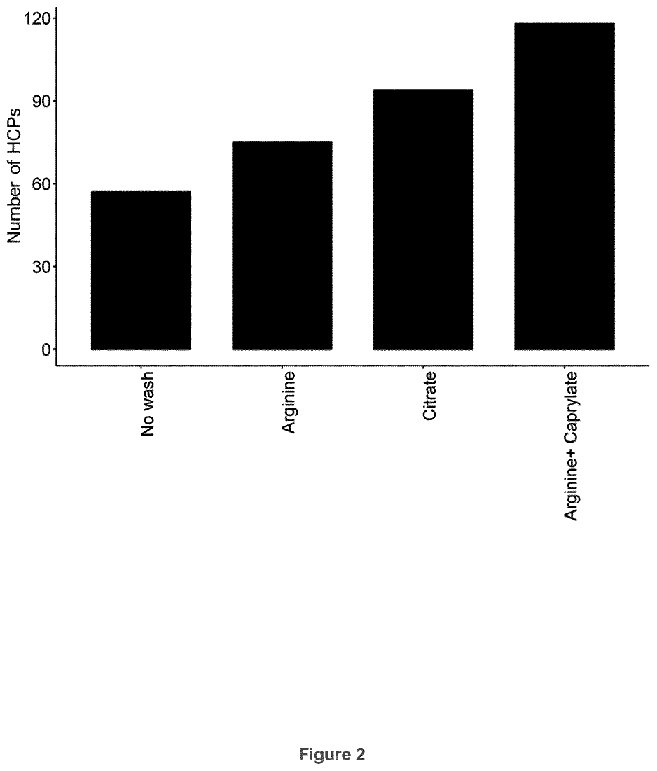

FIG. 3. Number of identified HCP protein groups processed with either a global HCP database or the in-house HCP database, using the method of the invention.

### DESCRIPTION OF THE INVENTION

The present invention solves the above identified need by providing a new method for the analysis, i.e. detection and quantification, of host cell proteins in a recombinant antibody sample that also allows for the assessment of HCP removal during protein purification.

In a first aspect, the present invention refers to a method for the detection of residual host cell proteins (HCP) in a recombinant protein sample comprising:
a) An affinity chromatography step, wherein the flow-through fraction is recovered
b) Tryptic digestion under native conditions of the flow-through fraction recovered from a), followed by
c) Liquid chromatography coupled with tandem mass spectrometry (LC-MS/MS) wherein the liquid chromatography is reversed-phase liquid chromatography performed in a multistep gradient of charge enhancer concentration,
d) Comparison of the results from said LC-MS/MS with an HCP database.

In a second aspect, the present invention refers to a label-free quantification method for residual HCPs in a recombinant protein sample comprising:
a) An affinity chromatography step, wherein the flow-through fraction is recovered
b) Tryptic digestion under native conditions of the flow-through fraction recovered from a), followed by
c) Liquid chromatography coupled with tandem mass spectrometry (LC-MS/MS) wherein the liquid chromatography is reversed-phase liquid chromatography performed in a multistep gradient of charge enhancer concentration,
d) Comparison of the results from said LC-MS/MS with an HCP database, and
e) Quantification of HCPs.
   Quantification of HCP by label free methods may be performed by available techniques such as spectral counting or intensity based measurements such as via Hi3 label free quantification. In a particular embodiment of the second aspect of the invention, the quantification of HCPs is performed via Hi3.

Affinity chromatography is a separation method based on a specific binding interaction between an immobilized ligand and its binding partner. In the context of the current invention, the affinity chromatography matrix is capable of binding the biotechnology product. Therefore, the flow through fraction recovered from the affinity chromatography according to the method of the invention is enriched in HCPs.

The skilled artisan is aware of different affinity chromatography matrices suitable for recombinant proteins, for example immobilized metal affinity chromatography has been used for both recombinant protein and nucleic acid purification, or enzyme/substrate interaction and enzyme/inhibitor interactions have been used for recombinant protein separations.

In a particular embodiment of the invention the recombinant protein is a recombinant antibody.

The affinity chromatography matrix is capable of binding the antibody, preferably through an Fc region or VH3 domain of the antibody.

In one embodiment, said affinity chromatography matrix is selected from: a protein A chromatography matrix, a protein G chromatography matrix and a protein L chromatography matrix.

There are many affinity chromatography materials containing protein A, protein G or protein L available to the skilled artisan, such as for example MabSelect® (GE Healthcare), Absolute® (Novasep), Captiv A® (Repligen), Praesto AP (Purolite) or Amsphere® (JSR) Nab™ Protein (ThermoFisher), or Pierce™ Protein (ThermoFisher).

Buffers suitable for use as wash and elution buffers in protein A chromatography are readily available in the art, and may be chosen by way of non-limiting examples from among phosphate buffered saline (PBS), Tris, histidine, acetate, formate, citrate buffers, or MES (2-(N-morpholino)ethanesulphonic acid Imidazole), BES (N,N-(bis-2-hydroxyethyl)-2-aminoethanesulphonic acid), MOPS (3-(N-morpholino)-propanesulphonic acid), or HEPES (N-2-hydroxyethylpiperazine-N′-2-ethanesulphonic acid) buffers.

In a particular embodiment of the method of the invention, the affinity chromatography step comprises:
i) contacting the sample with an affinity chromatography matrix
ii) Recovering the flow through from i)
iii) Applying a wash buffer to the affinity chromatography matrix and recovering the flow through
iv) Combining the flow through from steps ii) and iii).

In a particular embodiment of the method of the invention, the wash buffer used during affinity chromatography comprises a benzoate salt, benzyl alcohol, arginine, sodium caprylate, citrate salts, and/or quaternary ammonium salts such as tetra methyl ammonium chloride, tetra butyl ammonium. In a further preferred embodiment said wash buffer comprises, arginine, sodium caprylate or a combination of both.

In a further particular embodiment of the invention, the wash buffer comprises 0.2M arginine to 1M arginine, 0.2M arginine to 0.8M arginine, 0.2M arginine to 0.6M arginine, 0.2M arginine to 0.5M arginine, or 0.2M arginine to 0.4M arginine, preferably 0.3M arginine.

There are different sources of arginine available to a person of skill in the art. In the context of the present invention arginine and L-arginine are used interchangeably. Typically, L-arginine is available as a free base, or as a salt such as L-arginine monohydrochloride.

In a particular embodiment of the invention, the wash buffer comprises 0.05M sodium caprylate to 0.15M sodium caprylate, 0.08M sodium caprylate to 0.12M sodium caprylate, 0.09M sodium caprylate to 0.11M sodium caprylate, preferably 0.1M sodium caprylate.

In a further particular embodiment of the invention, the wash buffer has a pH of 7 to 9, 7.2 to 9, 7.3 to 9, 7.4 to 9, 7.5 to 9, 7.6 to 9, 7.7 to 9, 7.8 to 9, 7.8 to 8.8, 7.8 to 8.6, 7.9 to 8.6, or 8.0 to 8.5.

Trypsin is a serine protease that cleaves proteins into peptides with an average size of 700-1500 daltons, which is in the ideal range for MS detection. It is highly specific, cutting at the carboxyl side of arginine and lysine residues, making the resulting C-terminal arginine and lysine peptides charged, and hence detectable by MS.

As a person of skill in the art would be aware, an alternative to trypsin is the use of a mixture of trypsine and Lysine-C protease that may sometimes be considered advantageous as the lysine C protease may digest lysine cleavages missed by the trypsin in the presence of residual trypsin inhibitor impurities.

In a particular embodiment of the method of the invention, the tryptic digestion is performed with trypsin or a mixture of trypsin and Lysine-C protease. Mixtures of trypsin and Lysine-C protease are readily available from commercial sources such as for example Trypsin/Lys-C Mix from Promega.

In a particular embodiment of the invention, the tryptic digestion is performed with an enzyme to antibody ratio of 1/250 to 1/2000, or 1/250 to 1/1500, or 1/250 to 1/1000, or 1/250 to 1/750, or 1/250 to 1/500.

The terms “digestion under native conditions” or “native digestion” are used interchangeably, and in particular are used to differentiate from “traditional” or “normal” protein digestion, where the protein is denatured prior to digestion, i.e. prior to exposure to digestive enzymes such as trypsin.

In a particular embodiment of the method of the invention, the tryptic digestion is followed by treatment with a mild reducing agent selected from di-thiotreitol (DTT) or tris(2-carboxyethyl)phosphine (TCEP).

In a particular embodiment of the method of the invention, the mild reducing agent is present at a concentration of between 1 mM and 7 mM, preferably 1.5 mM to 6 mM, 1.5 mM to 5 mM, 1.5 mM to 4 mM, 2 mM to 4 mM, 2.5 mM to 3.5 mM, or 3 mM.

The resulting complex mixture of peptides is analyzed by reversed-phase liquid chromatography (RP-LC) coupled to tandem mass spectrometry (MS/MS). Identification of peptides and subsequently proteins is completed by matching peptide fragment ion spectra to a host cell protein database.

Reversed-phase liquid chromatography is an elution procedure used in liquid chromatography in which the mobile phase is significantly more polar than the stationary phase, e.g. a microporous silica-based material with chemically bonded alkyl chains.

In a particular embodiment of the method of the invention the reversed-phase liquid chromatography is ultra performance liquid chromatography or high pressure liquid chromatography. In a preferred embodiment of the method of the invention the reversed-phase liquid chromatography is ultra performance liquid chromatography.

In a particular embodiment of the method of the invention, the reversed-phase liquid chromatography is performed under heated conditions, preferably at 45° C. to 65° C., 55° C. to 65° C., or 60° C. to 65° C. In a particularly preferred embodiment of the method of the invention, the reversed-phase liquid chromatography is performed at 65° C.

Suitable columns for performing a reversed-phase liquid chromatography for peptide separation typically include C4, C8 or C18 columns. It is common knowledge in the art that the terms C4, C8 or C18 refer to the number of carbon atoms present in the alkyl chains of the stationary phase.

In a particular embodiment of the method of the invention the reversed-phase liquid chromatography is performed using a C18 column.

As a skilled person would know, mass spectrometry is one of the most popular methods used for proteomics analysis. For protein and peptide analysis by MS there are practical benefits to enhance analyte charge as it may facilitate further structural analysis and increase resolution and accuracy by increasing the efficiency of peptide ionisation and the coalescence of ion current into fewer charged states.

In a particular embodiment of the method of the invention, the reversed-phase liquid chromatography is performed in the presence of a charge enhancer. In a preferred particular embodiment, the charge enhancer comprises di-methyl sulfoxide (DMSO), meta-nitrobenzyl alcohol (m-NBA), ortho-nitroanisole (o-NA), ethylene carbonate (EC), propylene carbonate (PC), or sulfolane.

In a particular embodiment of the method of the invention, the multistep gradient of charge enhancer concentration is either an increasing gradient or a decreasing gradient.

In a particular embodiment, the gradient of charge enhancer concentration is 5% to 0.9%, 4% to 0.9%, 3.5% to 0.9%, 3% to 0.9%, 2.5% to 0.9%, 2% to 0.9% or 1.5% to 0.9%.

In a further embodiment, the gradient of charge enhancer is 1.5% to 0.9% DMSO.

The separation of peptides resulting from protein digestion followed by tandem mass spectrometry analysis, is frequently referred to as a shotgun-MS approach. There are currently two broad approaches toward generating such MS proteomic data: data-dependent acquisition and data-independent acquisition. In tandem mass spectrometry, the data dependent acquisition approach only puts forward certain peptides generated during the first cycle of MS for fragmentation during the second cycle, while with the data-independent approach, all peptides generated during the first MS cycle can be fragmented in the second round. During data-dependent acquisition, the measured spectra are compared with those of an established database, whereas in data-independent acquisition, the multiplexed nature of the MS spectra required deconvolution and hence search methods relying on a database cannot be applied directly.

In a particular embodiment of the invention, the mass spectrometry is performed using data dependent acquisition.

In a further particular embodiment of the invention, the HCP database is an HCP database based on a specific HCP database originating from the same host cell line used to produce the recombinant protein. The HCP database can be created using a null cell line, i.e. a cell line that does not contain the gene expressing the recombinant protein, or a cell line expressing the recombinant protein. In a particular embodiment the HCP database originates from the combination of the recombinant protein expressing host cell line and the same host cell line without the recombinant protein expressing gene.

A frequent database used for evaluating the performance of methods, instrument performance and molecular characterization of monoclonal antibodies is the NIST monoclonal antibody (NISTmAb) reference material RM 8671 (https://www.nist.gov/programs-projects/nist-monoclonal-antibody-reference-material-8671) created based on the characterization of a single IgG1k. It is known that NISTmAb contains HCPs in the range of 100-300 ppm.

However, by generating an HCP database using the same host cell as was used to produce the recombinant antibody, the comparison with the MS spectra obtained using the method of the invention should be more reliable. Furthermore, when generating an HCP database it is possible to improve sensitivity by removing redundant entries and hence better defining the of HCPs to be characterized.

In a particular method according to the invention, the HCP database comprises 3800 and 5000 protein sequences, 4000 to 4800 protein sequences, 4200 to 4600 protein sequences, 4300 to 4500 protein sequences.

A recombinant protein, manufactured for large-scale commercial purposes can be produced by culturing prokaryotic (bacterial) or eukaryotic host cells transfected with one or more expression vectors encoding the recombinant protein.

In the context of the invention as a whole, suitable eukaryotic host cells are mammalian host cells (also named mammalian cells) and include Chinese Hamster Ovary (CHO cells), lymphocytic cell lines, e.g., NSO myeloma cells and SP2 cells, COS cells, myeloma or hybridoma cells. In a preferred embodiment, the mammalian cell is a CHO cell. Suitable types of CHO cells may include CHO-K1, CHOK1-SV, dhfr-CHO, such as CHO-DG44, CHO-DXB11, CHO-DXB1, or yet CHO-S cells.

Mammalian cells may be cultured in any medium that will support their growth and expression of the antibody, preferably the medium is a chemically defined medium that is free of animal-derived products such as animal serum and peptone. There are different cell culture mediums available to the person skilled in the art comprising different combinations of vitamins, amino acids, hormones, growth factors, ions, buffers, nucleosides, glucose or an equivalent energy source, present at appropriate concentrations to enable cell growth and protein production. Additional cell culture media components may be included in the cell culture medium at appropriate concentrations at different times during a cell culture cycle that would be known to those skilled in the art.

Mammalian cell culture can take place in any suitable container such as a shake flask or a bioreactor, which may or may not be operated in a fed-batch mode depending e.g. on the scale of production required. These bioreactors may be either stirred-tank or air-lift reactors. Various large scale bioreactors are available with a capacity of more than 1,000 L to 50,000 L, preferably between 5,000 L and 20,000 L, or to 10,000 L. Alternatively, bioreactors of a smaller scale such as between 2 L and 100 L may also be used to manufacture an antibody according to the method of the invention.

An antibody or antigen-binding fragment thereof that can be manufactured in accordance with the methods of the present invention is typically found in the supernatant of a mammalian host cell culture, typically a CHO cell culture. For CHO culture processes wherein the protein of interest, such as an antibody or antigen-binding fragment thereof, is secreted in the supernatant, said supernatant is collected by methods known in the art, typically by centrifugation. For avoidance of doubt, supernatant denotes the liquid lying above the sedimented cells resulting from the centrifugation of the cell culture.

The supernatant is typically filtered to remove charged particles, remaining cell metabolites and cell debris, and the resulting fluid is typically referred to as a clarified cell culture fluid that will then be further processed with a number of steps, typically including 2 or 3 chromatography steps and ultrafiltration/diafiltration steps in order to obtain the purified antibody or antigen-binding fragment thereof.

During this further purification or downstream purification process, residual impurities including host cell proteins, protein aggregates and degradation products, will also be removed.

In this context it may be of interest to measure the final concentration of HCPs in the purified antibody sample (typically referred to as drug substance) to ensure adequate clearance, but it may also be of interest to perform this measurement during the purification steps to improve control of the overall process.

Therefore, in a particular embodiment of the method of the invention the antibody sample is obtained from the final purified drug substance. And in an alternative embodiment of the method of the current invention, the sample is obtained from a step in the purification stream of said recombinant antibody.

In a particular embodiment of the method of the invention, the antibody sample is a purified or partly purified antibody sample.

### Definitions

The term “antibody” or “antibodies” as used herein refers to monoclonal or polyclonal antibodies. The term “antibody” or “antibodies” as used herein includes but is not limited to recombinant antibodies that are generated by recombinant technologies as known in the art. “Antibody” or “antibodies” include antibodies' of any species, in particular of mammalian species; such as human antibodies of any isotype, including IgD, IgG₁, IgG₂ₐ, IgG_{2b}, IgG₃, IgG₄ IgE and antibodies that are produced as dimers of this basic structure including IgGA₁, IgGA₂, or pentamers such as IgM and modified variants thereof, non-human primate antibodies, e.g. from chimpanzee, baboon, rhesus or cynomolgus monkey; rodent antibodies, e.g. from mouse, or rat; rabbit, goat or horse antibodies; and camelid antibodies (e.g. from camels or llamas such as Nanobodies™) and derivatives thereof; or of bird species such as chicken antibodies or of fish species such as shark antibodies. The term “antibody” or “antibodies” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody performance. Humanization reduces the immunogenicity of non-human antibodies in humans, thus facilitating the application of antibodies to the treatment of human disease. Humanized antibodies and several different technologies to generate them are well known in the art. The term “antibody” or “antibodies” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies with specificity against a particular antigen upon immunization of the transgenic animal carrying the human germ-line immunoglobulin genes with said antigen. Technologies for producing such transgenic animals and technologies for isolating and producing the human antibodies from such transgenic animals are known in the art. Alternatively, in the transgenic animal, e.g. mouse, only the immunoglobulin genes coding for the variable regions of the mouse antibody are replaced with corresponding human variable immunoglobulin gene sequences. The mouse germline immunoglobulin genes coding for the antibody constant regions remain unchanged. In this way, the antibody effector functions in the immune system of the transgenic mouse and consequently the B cell development is essentially unchanged, which may lead to an improved antibody response upon antigenic challenge in vivo. Once the genes coding for a particular antibody of interest have been isolated from such transgenic animals the genes coding for the constant regions can be replaced with human constant region genes in order to obtain a fully human antibody. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” or “antibodies” as used herein, also refers to an aglycosylated antibody.

The term “antibody” or “antibodies” as used herein not only refers to untruncated antibodies of any species, including from human (e.g. IgG) and other mammalian species, but also refers to an antibody fragment. A fragment of an antibody comprises at least one heavy or light chain immunoglobulin domain as known in the art and binds to one or more antigen(s). Examples of antibody fragments according to the invention include Fab, Fab′, F(ab′)2, and Fv and scFv fragments; as well as diabodies, triabodies, tetrabodies, minibodies, domain antibodies (dAbs), such as single domain antibodies (sdAbs), V_{H}H and V_{NAR} fragments, single-chain antibodies, bispecific, trispecific, tetraspecific or multispecific antibodies formed from antibody fragments or antibodies, including but not limited to Fab-Fv or Fab-Fv-Fv constructs. Antibody fragments as defined above are known in the art.

### REFERENCES

1. Wang, X., Hunter, A. K., and Mozier, N. M. Biotechnol. Bioeng., 2009, vol. 103, no. 3, pp. 446-458.
2. Singh, S. K., Mishra, A., Yadav, D., Budholiya, N., and Rathore, A. S. Biotechnol. Prog., 2020, vol. 36, no. 2, p. e2936.
3. Jones, M., Palackal, N., Wang, F., Gaza-Bulseco, G., Hurkmans, K., Zhao, Y., Chitikila, C., Clavier, S., Liu, S., Menesale, E., Schonenbach, N. S., Sharma, S., Valax, P., Waerner, T., Zhang, L., and Connolly, T. Biotechnol. Bioeng., 2021, vol. 118, no. 8, pp. 2870-2885.
4. Bracewell, D. G., Francis, R., Smales, C. M. Biotechnol. Bioeng., 2015, vol. 112, pp. 1727-1737.
5. Ahrné, E., Molzahn, L., Glatter, T., and Schmidt, A. Proteomics, 2013, vol. 13, no. 17, pp. 2567-2578.
6. Kreimer, S., Gao, Y., Ray, S., Jin, M., Tan, Z., Mussa, N. A., Tao, L., Li, Z., Ivanov, A. R., and Karger, B. L. Anal. Chem., 2017, vol. 89, no. 10, pp. 5294-5302.
7. Meier, F., Geyer, P. E., Virreira Winter, S., Cox, J., and Mann, M. Nat. Methods, 2018, vol. 15, no. 6, pp. 440-448.
8. Huang, L., Wang, N., Mitchell, C. E., Brownlee, T., Maple, S. R., and De Felippis, M. R. Anal. Chem., 2017, vol. 89, no. 10, pp. 5436-5444.
9. Johnson, R. O., Greer, T., Cejkov, M., Zheng, X., and Li, N. Anal. Chem., 2020, vol. 92, no. 15, pp. 10478-10484.
10. Silva, J. C., Gorenstein, M. V., Li, G. Z., Vissers, J. P., and Geromanos, S. J. Mol. Cell. Proteomics, 2006, vol. 5, no. 1, pp. 144-156.

### EXAMPLES

### Materials

From readily available batches of in-house UCB development products, a null (non-product expressing) harvest cell culture fluid (HCCF), an upstream HCCF of a genetically engineered dihydrofolate reductase-deficient (DG44) CHO cell line producing recombinant full length IgG antibody, and the corresponding purified mAb were obtained. Tris hydrochloride 1M (Tris-HCl, pH8.0) buffer, Sodium Deoxycholate (SDC), Pierce high-pH reversed-phase peptide fractionation kit, trypsin/Lys-C protease mix, dithiothreitol (DTT), Nab protein-A plus spin columns, Nab protein-L plus spin columns, triethylammonium bicarbonate (TEAB), iodoacetamide (IAM), dimethyl sulfoxide (DMSO), 3K Da and 100K Da protein concentrators PES MWCO, Acclaim PepMap 100 C18 (100 Å, 3 μm, 1 mm×150 mm) column, were purchased from Thermo Scientific, Waltham, MA, USA. Quan-Recovery vials, MassPREP Bovine Serum Albumin (BSA, SwissProt P02769) digestion standard, MassPREP Alcohol Dehydrogenase (ADH, SwissProt P00330) digestion standard, MassPREP Phosphorylase b (PYGM, SwissProt P00489) digestion standard, MassPREP Enolase (ENO1, SwissProt P00924) digestion standard, RapiGest SF Surfactant, and ACQUITY UPLC CSH C18 column (130 Å, 1.7 μm, 1 mm×150 mm) were purchased from (Waters, Milford, MA, USA). Sodium caprylate, sodium citrate, citric acid, sodium hydroxide, sodium chloride, and arginine hydrochloride were purchased from Sigma-Aldrich, Overijse, Belgium. Formic acid (FA), trifluoroacetic acid (TFA), acetonitrile (ACN), and LC-MS grade water from Biosolve, Valkenswaard, Netherlands.

### Sample Pre-Treatment

### mAb Normal Digestion

1 mg of mAb sample was diluted with 50 mM TEAB and denatured with 0.1% RapiGest. Sample was then reduced with 10 mM DTT for 60 min at 60° C. and carboxymethylated by 20 mM IAM for 30 min in the dark at room temperature. Next, the alkylated sample was digested with trypsin/Lys-C mix at 1:20 enzyme to protein ratio and incubated at 37° C. overnight. The reaction was stopped with 1% TFA, kept for 20 min and centrifuged. Supernatant was acidified and dried using a speed vacuum concentrator. Dried sample was dissolved in formic acid (FA), acetonitrile (ACN), water (0.1:2:98, v/v/v), spiked with the four MassPREP proteins and transferred into Quan-recovery vial before LC-MS/MS analysis.

### mAb Native Digestion

A modified native digestion approach of Huang et al [8] was adopted. 1 mg mAb sample was diluted using 50 mM TEAB. Sample was then digested with trypsin/Lys-C protease mix (1:500 ratio) at 37° C. overnight. Subsequently, disulphide bonds were reduced with 3 mM DTT and incubated for 10 min at 90° C. The resulting digest was centrifuged at 15000×g for 2 min. Supernatant was acidified and dried using a speed vacuum concentrator. Dried sample was dissolved in FA, ACN, water (0.1:2:98, v/v/v), spiked with the four MassPREP proteins and RTC mixture and transferred into Quan-recovery vial before LC-MS/MS analysis.

### mAb Affinity Depletion—Normal and Native Digestion

Protein-A and protein-L beaded agarose resins were used. For each, 10 mg of mAb sample was diluted in loading buffer (50 mM Tris.HCl, pH 7.0). Resins were first equilibrated and then samples were applied. The flow through was collected. To study the effect of wash solutions on the recovery of HCPs during affinity depletion, different wash strategies were investigated. No wash (loading buffer), wash 1 (50 mM Tris.HCl, 0.5M sodium chloride, 0.3M arginine, pH8.5), wash 2 (0.1% M citric acid, 0.5M NaCl, pH5.5), and wash 3 (50 mM Tris.HCl, 0.1M sodium caprylate, 0.3M arginine, pH8.0) were applied. The flow-through and wash solution from each experiment were pooled together and buffer-exchanged into 50 mM TEAB using 3K Da MWCO concentrators. Following the previously described normal or native digestion protocols, the buffer-exchanged samples were either denatured with RapiGest or digested directly.

### HCCF and mAb Offline High-pH Fractionation

HCCF aliquots (null and upstream), filtered through 0.2 m syringe filters, and purified mAb samples were all diluted with 50 mM TEAB before being denatured, reduced, carboxymethylated, digested, acidified, and centrifuged according to the normal digestion protocol described previously. The offline high-pH fractionation kit was used to fractionate the supernatants. Elution solutions containing 0.1% triethylamine and ramped percentages of ACN ranging from 5% to 50% were used to collect 8 fractions from each sample. Fractions were then dried in vacuum, reconstituted in 0.1% FA, and spiked with the four MassPREP proteins and RTC mixture and transferred into Quan-recovery vial before LC-MS/MS analysis.

### Reversed-Phase (RP)-LC-MS/MS for HCP Analysis

Peptides were separated at 65° C. on an Acclaim PepMap 100 C18 column using a Thermo Fisher Scientific Ultimate3000 UHPLC System with micro-LC setup. Samples were injected at 50 μl/min flow rate. Mobile phase A consisted of 1.5% DMSO, 0.1% FA in water and mobile phase B consisted of 0.1% FA in ACN. DMSO was added to mobile phase A as an organic modifier to increase the efficiency of peptide ionisation and the coalescence of ion current into fewer charge states. To improve peak capacity, a multistep gradient of 1% to 40% B in 160 min was applied, followed by a 5-min column wash at 80% B and column re-equilibration for 10 min at 1% B. The UHPLC system was coupled with an Exploris 480 mass spectrometer (Thermo Fisher Scientific). Tandem MS analysis was performed using DDA with the following settings: MS scans had an m/z range of 360-1300, a resolution of 120K full width half maximum (FWHM), an automatic gain control (AGC) target of 1E6, and a maximum injection time (MIT) of 200 ms. Top-40 most abundant ions were selected from every MS survey scan and isotopic exclusion was used. MS/MS was performed on ions exceeding 5000 counts with charge states ranging from +2 to +4. Higher energy collisional dissociation (HCD) was carried out with a normalised energy of 28% and followed by ion trap analysis. An isolation window of 2 m/z was used, and the dynamic exclusion duration was set to automatic. MS/MS scans had a 15K resolution (FWHM), an AGC target of 2E5, and 50 ms MIT.

### Construction of In-House Database

The resulting MS/MS data from the high-pH fractionated samples were searched against a customised database with 78,119 protein sequences containing all Critecutulus griseus entries from Uniprot.org (Swiss-Prot and TrEMBL annotations included), the mAb heavy and light chains, and common contaminants. A de-novo sequencing assisted database search hybrid program Byos v4.1 (Protein Metrics, San Carlos, CA) was used for the search. Search parameters included mass tolerance as 10 ppm for precursors and 20 ppm for fragments, semi specific peptide termini with ≤2 missed cleavages, automatic peptide score cut and protein false discovery rate (FDR) cut-off at 2%. Cysteine carboxymethylation was considered as a fixed modification, methionine or tryptophan oxidation as common variable modifications and asparagine deamidation as a rare variable modification. A maximum of 1 common and 1 rare modification were allowed. Protein hits were adjusted to |Log Prob| (log base 10 of the protein p-value) score at least 2.0 lower than the top decoy protein score and PSM filtering was deferred until after protein assembly. Common protein contaminants were excluded, and only the doubly and triply charged peptides were considered. In total, 4380 proteins were identified and consolidated to build an in-house database. The sequences of these proteins were used to generate a database in FASTA format.

### Peptide and Protein Identification Using In-House Database

The resulting MS/MS data from analysed samples were searched by Byos against the in-house database (4380 protein sequences), the Immunoglobulin G-binding protein A sequence, the four standard protein digests of the MassPREP (P02769, P00330, P00489 and P00924), the mAb heavy and light chains, common contaminants. Search parameters included mass tolerance as 10 ppm for precursors and 20 ppm for fragments, fully specific peptide termini with ≤2 missed cleavages, automatic peptide score cut and protein FDR cut-off at 2%. Methionine or tryptophan oxidation were considered common variable modifications. Asparagine deamidation, N-terminal glutamine formation of pyroglutamate, N-terminal acetylation, and serine dehydration were considered rare variable modifications. A maximum of 1 common and 1 rare modification were allowed. Protein hits were adjusted to |Log Prob| score at least 2.0 lower than the top decoy score. PSM filtering was deferred until after protein assembly. Common protein contaminants were excluded, and only the doubly and triply charged peptides were considered. Further peptide/protein filtering was performed by eliminating peptides that had less than six amino acids, and all single-spectrum protein hits. Features that are missing in a replicate but are correctly assigned in other replicates were inferred by Byos, thereby reducing missing value and improving our subsequent differential abundance analysis.

### Hi3 Quantification

All peptides that passed identification filtering were exported along with their respective MS1 XIC peak areas. All intensities were normalised by dividing them by the total intensity per replicate and multiplying them by the mean of the three replicates' total intensities. Prior to Hi3 LFQ measurement, all modified peptides and those with a coefficient of variation greater than 25% within their triplicates were eliminated. A universal signal-response curve was produced by plotting the sum of the average MS1 intensities of the top three peptides for all the four proteins of MassPREP against their concentrations (in fmoles) [10]. A linear curve fit was demonstrated and utilised to estimate individual HCP abundance (in moles) based on the sum of the average intensities of the top 3 peptides for each identified HCP. The quantity of each identified HCP was estimated (in ng/mg) using its molecular weight and the initial mAb amount.

### Results

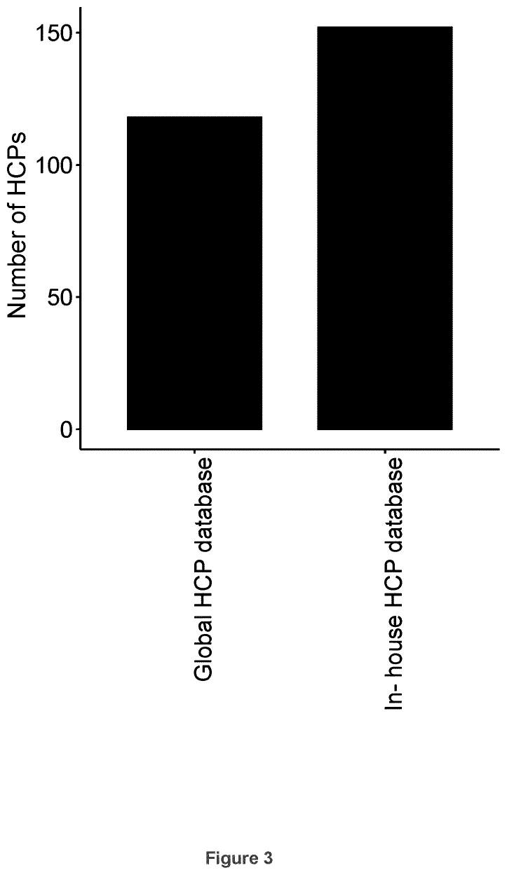

The MS/MS data of a purified mAb analyzed by the method of the invention was searched against the in-house HCP database (4380 protein sequences) and the global HCP database that contains all C. griseus entries (78119 protein sequences). An increase of 28% in the number of identified HCP protein groups (FIG. 3) and 16% in unique peptides was observed when using in-house database. This notable boost in scoring sensitivity achieved by using a low-redundancy database will enable more thorough and high-quality HCP profiling.

1-15. (canceled)

16. A method for detection of residual host cell proteins (HCP) in a recombinant protein sample comprising:
a) an affinity chromatography step, wherein the flow-through fraction is recovered;
b) tryptic digestion under native conditions of the flow-through fraction recovered from a), followed by
c) liquid chromatography coupled with tandem mass spectrometry (LC-MS/MS), wherein the liquid chromatography is reversed-phase liquid chromatography performed in a multistep gradient of charge enhancer concentration, and
d) comparison of the results from said LC-MS/MS with an HCP database.

17. The method according to claim 16, wherein the affinity chromatography step comprises:
i) contacting the sample with affinity chromatography matrix;
ii) recovering the flow through from i);
iii) applying a wash buffer to the affinity chromatography matrix and recovering the flow through; and
iv) combining the flow through from steps ii) and iii).

18. The method according to claim 16, wherein the recombinant protein is a recombinant antibody.

19. The method according to claim 16, wherein the affinity chromatography matrix is selected from protein A, protein G or protein L.

20. The method according to claim 17, wherein the wash buffer comprises arginine, sodium caprylate, citrate salts, and/or quaternary ammonium salts.

21. The method according to claim 16, wherein the tryptic digestion is performed with a mixture of trypsin and lysine-C protease.

22. The method according to claim 16, wherein the tryptic digestion is followed by treatment with a mild reducing agent selected from di-thiotreitol (DTT) or tris(2-carboxyethyl)phosphine (TCEP).

23. The method according to claim 16, wherein the reversed-phase liquid chromatography is ultra performance liquid chromatography.

24. The method according to claim 16, wherein the reversed-phase liquid chromatography is performed under heated conditions.

25. The method according to claim 16, wherein the reversed-phase liquid chromatography is performed using a C18 column.

26. The method according to claim 16, wherein the charge enhancer comprises di-methyl sulfoxide (DMSO), meta-nitrobenzyl alcohol (m-NBA), ortho-nitroanisole (o-NA), ethylene carbonate (EC), propylene carbonate (PC), or sulfolane.

27. The method according claim 26, wherein the gradient of charge enhancer is 5% to 0.9%, or 1.5% to 0.9% DMSO.

28. The method according to claim 16, wherein the mass spectrometry is performed using data dependent acquisition.

29. The method according to claim 16, wherein the HCP database is an HCP database based on an HCP database originating from the host cell line used for producing the antibody.

30. The method according to claim 16, wherein the antibody sample is a purified or partly purified antibody sample.

31. The method according to claim 24, wherein the reversed-phase liquid chromatography is performed at between 45° C. and 65° C.
